(19) United States
(12) Patent Application Publication
(10) Pub. No.: US 2010/0221321 A1
(43) Pub. Date: Sep. 2, 2010

(54) Apparatus And Methods For Delivering A Plurality Of Medicaments For Management Of Co-Morbid Diseases, Illnesses Or Conditions

(75) Inventors: Frederick H. MILLER, St. Petersburg, FL (US); James H. Ruble, Bountiful, UT (US); Gary D.E. Pierce, Kaysville, UT (US)

Correspondence Address: Davidson, Davidson & Kappel, LLC, 485 7th Avenue, 14th Floor, New York, NY, 10018, US

(73) Assignee: INNERCAP Technologies, Inc., St. Petersburg, FL (US)

(21) Appl. No.: 12/719,993

(22) Filed: Mar. 9, 2010

Related U.S. Application Data
(63) Continuation of application No. 10/902,261, filed on Jul. 30, 2004, now abandoned.
Application No. 10/902,261 is a continuation-in-part of application No. 10/804,224, filed on Mar. 19, 2004, now abandoned.
Application No. 10/804,224 is a continuation-in-part of application No. 10/786,563, filed on Feb. 26, 2004, now abandoned.
(60) Provisional application No. 60/451,150, filed on Feb. 27, 2003.

(51) Int. Cl. A61K 9/48; A61K 31/40; A61K 31/135; A61K 31/4439; A61K 31/4525; A61P 25/24; A61P 3/06

(52) U.S. Cl. 424/451; 514/423; 514/647; 514/342; 514/321

(57) ABSTRACT

A method and apparatus for delivering a plurality of medicaments in a single delivery vehicle for the management of co-morbid diseases, illnesses and conditions. The present invention provides a novel delivery process for many medicaments. Medicaments may be encapsulated and stored separately within a larger capsule until the time of ingestion, consumption, or the like. Benefits of the present invention include maintaining separation of distinct ingredients within a single capsule and the capability to control the time release of multiple ingredients within the capsule.

### CROSS-REFERENCE TO RELATED PATENT APPLICATIONS

This is a Continuation-in-part of U.S. patent application Ser. No. 10/804,224, filed Mar. 19, 2004, which is a Continuation-in-part of U.S. patent application Ser. No. 10/786,563, filed on Feb. 26, 2004, which claim priority of U.S. Provisional Patent Application Ser. No. 60/451,150, filed Feb. 27, 2003, entitled, Apparatus and Methods for Delivering a Plurality of Medicaments for Management of a Disease, Illness or Condition Affecting One or More Organ Systems, identifying Frederick H. Miller as the inventor, the contents of which are incorporated by reference herein in their entirety. This application is related to U.S. patent application Ser. No. 10/786,564, filed on Feb. 26, 2004, which claim priority of U.S. Provisional Patent Application Ser. No. 60/450,615, filed on Feb. 27, 2003, entitled, Apparatus and Methods for Delivering a Plurality of Medicaments for Management of Co-Morbid Diseases, Illnesses or Conditions, identifying Frederick H. Miller as the inventor, the contents of which are incorporated by reference herein in their entirety.

### FIELD OF THE INVENTION

The present invention relates to methods for the administration of a plurality of heterogenous chemical and biological compounds to animals and humans using a multi-compartment delivery system for treatment of separate conditions or diseases in one or more organ systems.

### BACKGROUND OF THE INVENTION

Administration of chemicals and biomolecules to animals and humans may first appear to be a simple undertaking, however, the contemplation, design, testing, and manufacture of elegant pharmaceutical products requires a thorough integration of medical and pharmacy principles.

The art and practice of pharmacy can be divided into four distinct divisions. Pharmacology is the study of interactions occurring between the pharmacologic agent, or medicament and specific targeted cells in the body. More specifically, the interaction between an active agent and a cellular receptor along with the resulting change in cell physiology is examined. Medicinal chemistry is largely concerned with the identification of naturally occurring and synthetic compounds which possess medicinal characteristics. Pharmacotherapeutics is the holistic application of pharmacy practice to specific pathologies, illnesses, and other body functions. Finally, Pharmaceutical science ascertains or regulates the composition of medicinal substances, and is largely directed to the development of new mechanisms for delivering chemicals and biomolecules into animals and humans. A sub-category of pharmaceutical science is called pharmacokinetics and sometimes generally referred to as biopharmaceutics.

A.D.M.E. is an acronym often used to describe the four essential components to pharmaceutical science: absorption, distribution, metabolism, and elimination, respectively. One way to differentiate between pharmacology and pharmaceutical science is that the former is primarily concerned with the effect of the medicament on the body, whereas, the latter is primarily concerned with the delivery and time-course of the medicament on its journey through the body.

In clinical applications, chemicals and biomolecules are often referred to as active ingredients or medicaments. Medicaments may include “pharmaceuticals, nutraceuticals, biotechnicals, vitamins, minerals and dietary supplements.” Oral administration is the most frequent route for delivery of medicaments. Medicaments may be orally administered in a variety of physical states, including, solid, liquid, dispersion, and gaseous forms. As appreciated, tablets and capsules are the most common vehicle for oral delivery of medicaments.

Frequently, a medical or surgical patient may receive a plurality of concurrent medicaments. Data has been accumulated to demonstrate that patients undergoing a surgical procedure may receive ten (10) or more medicaments during the surgery and the resulting surgical recovery period. Some patients who have undergone organ transplantation or who have contracted human immunodeficiency virus (HIV) may receive three (3) or more medicaments which require multiple administrations per day. HIV patients often receive many more than three (3) medicaments. These medicaments may be necessary for the treatment of several conditions occurring in a plurality of organ systems or they may be necessary to treat a single condition or some combination thereof.

In some cases, it may be desirous to combine a plurality of medicaments because of a synergistic interaction between a plurality of medicaments. This synergy may enhance the efficacy of one or more of the medicaments. Medicaments may be combined to increase the intensity of response or efficacy. A plurality of medicaments, in combination, may be homergic (i.e., elicit the same quality of effect). In many cases, a plurality of homergic medicaments may also be homonymic (i.e., interact with the same receptor). A plurality of homergic medicaments may be additive, supra-additive and infra-additive. A plurality of combined medicaments which do not produce the same quality of response may be called, heterergic. When heterergy is found to be a positive effect (i.e., at least one medicament enhances the response to another medicament), this may be called synergism and is sometimes called synergy.

In further cases, it may be desirous to combine a plurality of medicaments to decrease their individual dosages and possibility for toxicity. It may also be desirous to combine a plurality of medicaments to target the treatment of a disease, illness or condition from divergent angles. It may be desirous to combine a plurality of medicaments to minimize the side effects and adverse effects of one or more medicaments. It may be still further desirous to combine a plurality of medicaments to alter the pharmacokinetic characteristics of one or more medicaments. For example, alterations in the absorption, distribution, metabolism or elimination of one or more medicaments.

Fixed combinations of a plurality of medicaments have been generally disfavored due to any number of perceived disadvantages. These disadvantages may include, for example: (1) complicating the interpretation of safety and efficacy in therapeutic regimens, (2) there may be inter-patient differences to fixed combinations, (3) there may be difficulties in dosage titration, and (4) the delivery platforms for fixed combinations have generally been found to be uneconomical to produce.

On the other hand, fixed combinations of a plurality of medicaments may lead to several therapeutic advantages, including, for example, but not by way of limitation: (1) increasing patient compliance with therapy, (2) increasing efficacy by optimizing timing of medicaments, (3) minimization of side effects and adverse effects, (4) enhancement of pharmacokinetic characteristics of one or more medicaments in a fixed combination, (5) increased patient quality of life, (6) optimization of institutional resources by minimizing the amount of medicament administrations, and (7) minimizing patient length of stay in institutional facilities by optimizing therapy.

Prior art therapeutic technologies contain isolated examples of pharmaceutical formulations containing fixed combinations of medicaments. However, therapeutic technologies of the prior art teach a fixed combination, wherein a plurality of medicaments are placed into a single receiving chamber in the delivery formulation (i.e., no separation between the plurality of medicaments). In addition, those skilled in the art developed therapeutic technologies that contemplate the use of a multi-compartment (i.e., multiple-receiving chambered) delivery vehicle for treating Helicobacter pylori-induced peptic ulcer disease.

In view of the state of the technology as it exists today, generally, therapeutic apparatus and methods are needed to provide a plurality of medicaments for medical and surgical conditions, as well as maintenance of normal health function for delivery to animals and humans using a multi-chambered delivery apparatus. Such apparatus and methods for delivering a plurality of medicaments to animals and humans using a multi-chambered delivery apparatus are contemplated herein.

### SUMMARY OF THE INVENTION

A primary object of the present invention is to provide novel delivery apparatus and methods for affecting multiple organ systems in animals or humans using a plurality of medicaments delivered by a pharmaceutical formulation comprising a multi-chambered apparatus. Accordingly, the present invention provides novel delivery apparatus and administration techniques or methods aimed at affecting multiple organ systems in an animal or human using a plurality of medicaments. A delivery apparatus may be in any multi-chambered apparatus, but preferably in a capsular formulation. Thus, a plurality of medicaments may be encapsulated and stored separately within a larger capsule until the time of ingestion, consumption, or the like. Upon consumption, the capsule walls of one or more dividing walls of a capsule may dissolve to release their contents. Different methods of encapsulation may be used to deliver their respective contents, including but not limited to, dissolution, melting, ablation or biodegradation of the encapsulating wall. In certain embodiments and as contemplated herein, the medicaments retained in the multi-compartment capsule may actually diffuse through one or more of the encapsulating walls.

In accordance with one presently preferred embodiment of the present invention, an encapsulated product may be provided in several parts, including a primary capsule and one or more secondary capsules enclosed therein. In other presently preferred embodiments, a capsule may be provided having separate compartments or sections where ingredients may be stored and released at desired time intervals. More specifically, a process for encapsulating secondary smaller capsules in a larger primary capsule is contemplated hereinbelow. In one presently preferred embodiment, a process in accordance with the present invention may include the steps of
1. Providing a primary capsule with a receiving chamber, wherein, in certain preferred embodiments, the primary capsule may be provided in two parts, such as a body portion and a cap portion, which may be sealed together to retain ingredients contained therein;
2. Providing one or more secondary capsules having a dimensional size sufficient so that medicaments and the secondary capsule may be introduced within the receiving chamber of the primary capsule, wherein, in selected preferred embodiments, the secondary capsule may also be provided in two parts, such as a body portion and a cap portion, which may be sealed together to retain ingredients contained therein;
3. Filling the receiving chamber of a secondary capsule with one or more medicaments, wherein said medicaments may be the same or different from medicaments in other receiving chambers;
4. Sealing one or more medicaments within the secondary capsule;
5. Inserting the secondary capsule into at least a portion of the receiving chamber of the primary capsule;
6. Filling the body portion of the primary capsule, housing one or more secondary filled capsules, with one or more medicaments; and
7. Sealing one or more medicaments and one or more secondary capsules within a receiving chamber of the primary capsule.

### BRIEF DESCRIPTION OF THE DRAWINGS

The objects and features of the present invention will become more fully apparent from the following description, taken in conjunction with the accompanying drawings and appendices. Understanding that the Figures depict presently preferred embodiments of the present invention and are, therefore, not to be considered limiting of its scope, the invention will be described with additional specificity and detail through use of the accompanying drawings and appendices in which:

FIG. 1 is flow chart illustrating several preferred embodiments of the present invention, wherein said embodiments originating from five (5) proprietary products belonging to Pfizer, Incorporated;

FIG. 2 is flow chart illustrating several preferred embodiments of the present invention, wherein said embodiments originating from five (5) proprietary products belonging to Glaxo SmithKline, P.L.C;

FIG. 3 is flow chart illustrating several preferred embodiments of the present invention, wherein said embodiments originating from five (5) proprietary products belonging to Merck & Co;

FIG. 4 is flow chart illustrating several preferred embodiments of the present invention, wherein said embodiments originating from five (5) proprietary products belonging to AstraZeneca, P.L.C; and

FIG. 5 is flow chart illustrating several preferred embodiments of the present invention, wherein said embodiments originating from five (5) proprietary products belonging to Novartis, Incorporated.

### DETAILED DESCRIPTION OF THE PREFERRED EMBODIMENTS

It will be readily understood that the components of the present invention as detailed below, may be arranged and designed in a wide variety of different configurations and steps. Thus, the description herein is not intended to limit the scope of the invention, but is merely representative of certain presently preferred embodiments in accordance with the invention and its inherent inventive concepts. Those of ordinary skill in the art will, of course, appreciate that various modifications to the details herein may easily be made without departing from the essential characteristics of the invention, as described. Thus, the following information is intended only by way of example, and simply illustrates certain presently preferred embodiments consistent with the invention.

Appendix I contains a list of diseases, illnesses and conditions which are known to affect several organ systems and a list of medicaments which may be helpful in managing said diseases, illnesses and conditions, the contents of Appendix I being incorporated herein by reference in their entirety.

Referring to generally to Appendix I, it may be demonstrated that as medical and pharmacy knowledge has continued to expand exponentially, new medicaments, new classes of medicaments and new delivery technologies are becoming available for use in animals and humans who experience particular medical diseases, illnesses or conditions. A disease, illness, or condition may affect one or more organ systems in an animal or human. Organ systems may include, for example: (1) autonomic, (2) cardiovascular, (3) neurological, (4) gastro-intestinal, (5) respiratory, (6) renal system, (7) psychiatric, (8) endocrine, (9) gynecologic, (10) urologic, (11) immunologic, (12) bone and joint systems, (13) ear, nose, and throat, (14) dermatologic, (15) hematologic, (16) infectious defense and (17) nutrition and metabolism. In an animal or human who may be suffering from one disease, illness or condition, it is common to also be suffering from a disease, illness or condition affecting one or more of the other organ system(s). These concomitant diseases, illnesses or conditions occurring within a single animal or human are often labeled as “co-morbidities”, a term often shortened and referred to as “co-morbid.”

New medicaments and delivery technologies are providing patients and their health care practitioners with unprecedented therapeutic options in managing diseases, illnesses and conditions. In spite of this sophistication, there has been no effort to develop new methods of using fixed combinations of medicaments for therapy of co-morbid diseases, illnesses or conditions. Moreover, there has been no effort to develop new methods of using fixed combinations of medicaments for management of a single disease, illness or condition affecting one or more organ system(s). The aforementioned fixed combinations may include a plurality of medicaments, which may be newly discovered and developed, or have been known for sometime or some combination of medicaments thereof. In any regard, said fixed combinations have not previously been contemplated in the art.

The following examples will illustrate the invention in further detail. It will be readily understood that the various plurality of medicaments that may be introduced into the receiving chambers of the multi-compartment vehicles of the present invention, as generally described and illustrated in the Examples herein, are to be viewed as exemplary of the principles of the present invention, and not as restrictive to a particular structure or process for implementing those principles. Thus, the following more detailed description of the presently preferred embodiments of the methods, formulations, and compositions of the present invention, as represented in Examples I-V, is not intended to limit the scope of the invention, as claimed, but is merely representative of presently preferred embodiments of the invention.

### Example I

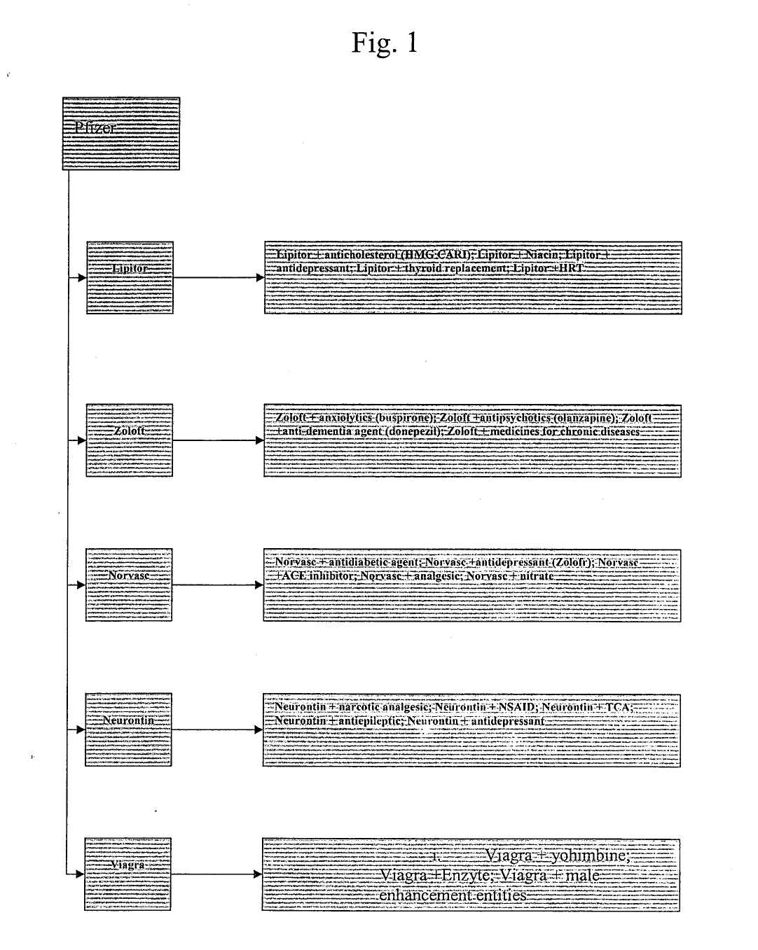

Referring to FIG. 1, in one presently preferred embodiment of the present invention, a plurality of medicaments are delivered in a single vehicle for management of co-morbid diseases, illnesses or conditions. More particularly, a plurality of medicaments comprising an HMG CoA Reductase inhibitor, for example, atorvastatin (LIPITOR® manufactured by Pfizer, Inc.), and a serotonin reuptake inhibitor, for example, sertraline (ZOLOFT® manufactured by Pfizer, Inc.), may be combined into a single delivery apparatus, for example, a multi-compartment capsule.

In the presently preferred embodiment, a therapeutically effective amount of atorvastatin (LIPITOR® manufactured by Pfizer, Inc.), may be introduced into a receiving chamber of a secondary capsule. The base and cap of the secondary capsule may then be sealed using any available conventional means readily known to those skilled in the art. The secondary capsule may then be introduced into a receiving chamber in a primary capsule. A therapeutically effective amount of sertraline (ZOLOFT® manufactured by Pfizer, Inc.) may be introduced into the receiving chamber of the primary capsule. The base and cap of the primary capsule may then be sealed using any available conventional means readily known to those skilled in the art.

A capsular format of the present invention may include the following composition:

Primary Capsule:

| | |
|---|---|
| sertraline (ZOLOFT ® manufactured by Pfizer, Inc.) | 100 mg |
| [dosage range 50-200 mg/day] | |

Secondary Capsule:

| | |
|---|---|
| atorvastatin (LIPITOR ® manufactured by Pfizer, Inc.) | 20 mg |
| [dosage range 10-80 mg/day] | |

The incorporation of time-release coatings to vary the release rates of the medicaments (e.g., sertraline (ZOLOFT® manufactured by Pfizer, Inc.) and atorvastatin (LIPITOR® manufactured by Pfizer, Inc.)) in the primary and secondary capsules, respectively, of the multi-compartment capsule may be used to target key time intervals or events when the body may be most able to utilize the named active ingredients. For example, HMG CoA Reductase Inhibitors are known to as much as a 70% increase in efficacy when administered in the evening hours. Thus, by varying the composition of the secondary capsular wall, release of the contents of the secondary capsule may be targeted to an optimal delivery time. A differential release between the primary capsule and secondary capsule may therefore optimize management of co-morbid conditions in a patient. The patient is able to take a single daily administration vehicle, which provides a plurality of medicaments for managing co-morbid conditions. Thus, the incorporation of time-release coatings in the encapsulation process when forming a multi-compartment capsule may be specifically designed to fit the needs and desires of numerous different users having similar conditions that are being targeted for treatment. It is intended, therefore, that the examples provided herein be viewed as exemplary of the principles of the present invention, and not as restrictive to a particular structure or method for implementing those principles.

### Example II

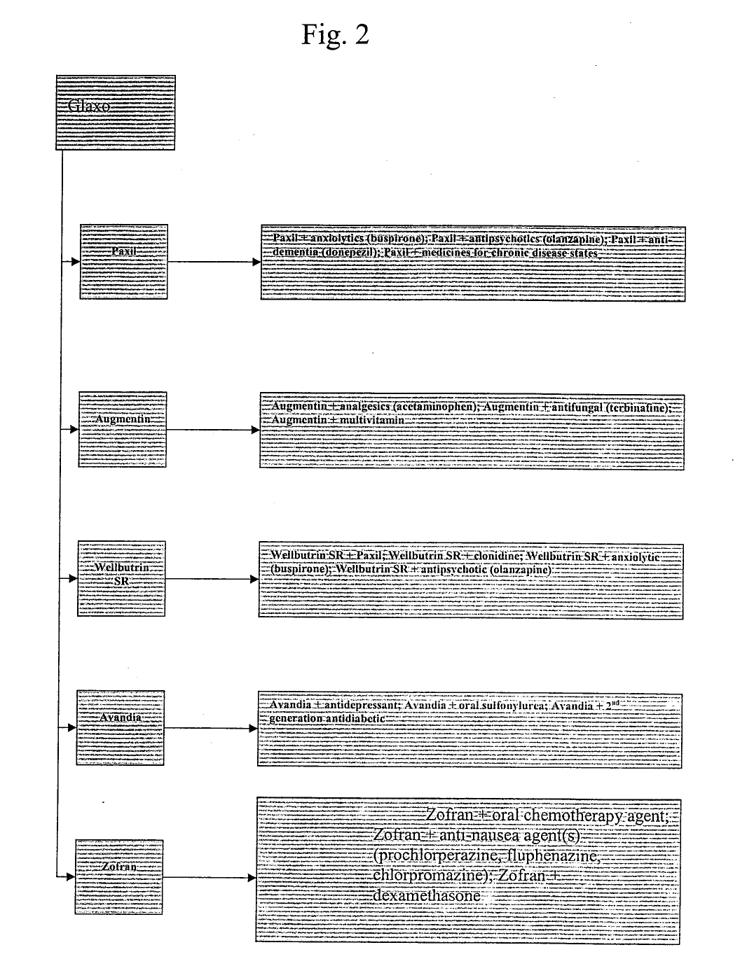

Referring to FIG. 2, in one presently preferred embodiment of the present invention, a plurality of medicaments are delivered in a single vehicle for management of co-morbid diseases, illnesses or conditions. More particularly, a plurality of medicaments comprising a thiazolidinedione derivative, for example, rosiglitazone (AVANDIA® manufactured by Glaxo SmithKline), and a serotonin reuptake inhibitor, for example, paroxetine (PAXIL® manufactured by Glaxo SmithKline) may be combined into a single delivery apparatus, for example, a multi-compartment capsule.

In the presently preferred embodiment, a therapeutically effective amount of paroxetine (PAXIL® manufactured by Glaxo SmithKline) may be introduced into a receiving chamber in a secondary capsule. The base and cap of the secondary capsule may then be sealed using any available means readily known to those skilled in the art. A therapeutically effective amount of rosiglitazone (AVANDIA® manufactured by Glaxo SmithKline) may be introduced into a receiving chamber in a primary capsule. The secondary capsule may then be introduced into a receiving chamber in a primary capsule. The primary capsule may then be sealed using any available means readily known to those skilled in the art.

A capsular format of the present invention may include the following composition:

Primary Capsule:

| | |
|---|---|
| rosiglitazone (AVANDIA ® manufactured by Glaxo | 4 mg |
| SmithKline) [dosage range 4-8 mg/day] | |

Secondary Capsule:

| | |
|---|---|
| paroxetine (PAXIL ® manufactured by Glaxo SmithKline) | 20 mg |
| [dosage range 20-60 mg/day] | |

The incorporation of time-release coatings to varying the release rates of the medicaments (e.g., rosiglitazone (AVANDIA® manufactured by Glaxo SmithKline) and paroxetine (PAXIL® manufactured by Glaxo SmithKline)) in the primary and secondary capsules, respectively, of the multi-compartment capsule may be used to target key time intervals or events when the body may be most able to utilize the named active ingredients. For example, paroxetine (PAXIL® manufactured by Glaxo SmithKline) is known to have sedating effects similar to antihistamines. Therefore, to minimize these effects on activities of daily living, paroxetine (PAXIL® manufactured by Glaxo SmithKline) is recommended for administration in the evening hours. Thus, by varying the composition of the secondary capsule wall, release of the contents of the secondary capsule may be targeted to an optimal time. A differential release between the primary capsule and secondary capsule may therefore optimize management of co-morbid conditions in a patient. The patient is able to take a single daily administration vehicle, which provides a plurality of medicaments for managing co-morbid conditions. Thus, the incorporation of time-release coatings in the encapsulation process when forming a multi-compartment capsule may be specifically designed to fit the needs and desires of numerous different users having similar conditions that are being targeted for treatment. It is intended, therefore, that the examples provided herein be viewed as exemplary of the principles of the present invention, and not as restrictive to a particular structure or method for implementing those principles.

### Example III

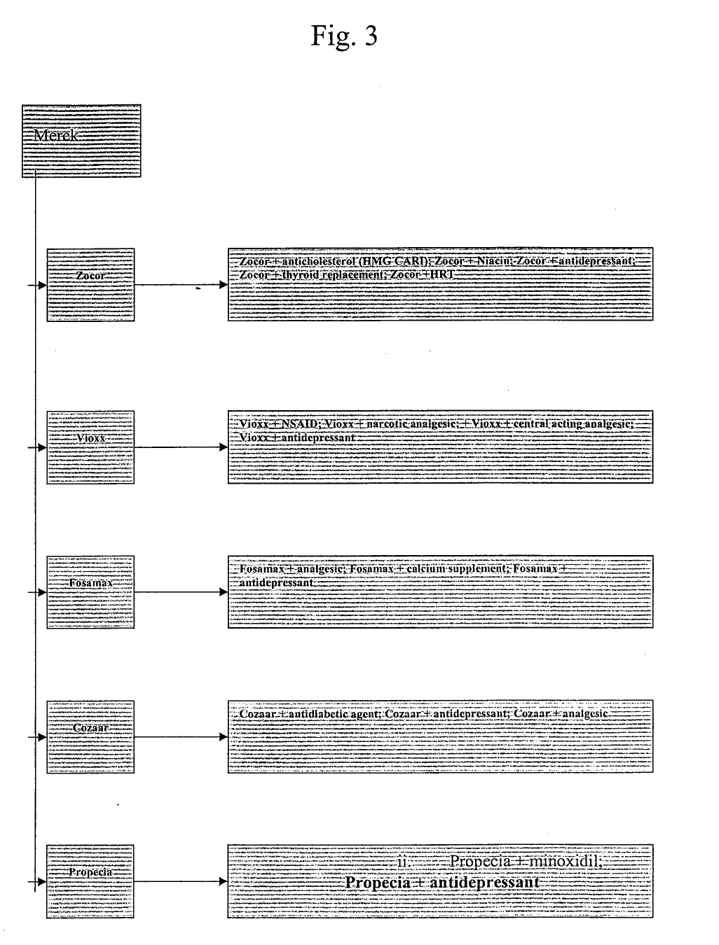

Referring to FIG. 3, in one presently preferred embodiment of the present invention, a plurality of medicaments are delivered in a single vehicle for management of co-morbid diseases, illnesses or conditions. More particularly, a plurality of medicaments comprising a bisphosphonate, for example, alendronate (FOSAMAX® manufactured by Merck & Co.) and an HMG CoA Reductase Inhibitor, for example, simvastatin (ZOCOR® manufactured by Merck & Co.) may be combined into a single delivery apparatus, for example, a multi-compartment capsule.

In the presently preferred embodiment, a therapeutically effective amount of simvastatin (ZOCOR® manufactured by Merck & Co.) may be introduced into a receiving chamber in a secondary capsule. The base and cap of the secondary capsule may then be sealed using any available conventional means readily known to those skilled in the art. The secondary capsule may then be introduced into a receiving chamber in a primary capsule. A therapeutically effective amount of alendronate (FOSAMAX® manufactured by Merck & Co.) may be introduced into a receiving chamber in a primary capsule. The base and cap of the primary capsule may then be sealed using any available conventional means readily known to those skilled in the art.

A capsular format of the present invention may include the following composition:

Primary Capsule:

| | |
|---|---|
| alendronate (FOSAMAX ® manufactured by Merck & Co.) | 5 mg |
| [dosage range 5-10 mg/day] | |

Secondary Capsule:

| | |
|---|---|
| simvastatin (ZOCOR ® manufactured by Merck & Co.) | 40 mg |
| [dosage range 5-80 mg/day] | |

The incorporation of time-release coatings to varying the release rates of the medicaments (e.g., alendronate (FOSAMAX® manufactured by Merck & Co.) and simvastatin (ZOCOR® manufactured by Merck & Co.)) in the primary and secondary capsules, respectively, of the multi-compartment capsule may be used to target key time intervals or events when the body may be most able to utilize the named active ingredients. For example, HMG CoA Reductase Inhibitors are known to as much as a 70% increase in efficacy when administered in the evening hours. Therefore, to maximize its effects, simvastatin (ZOCOR® manufactured by Merck & Co.) is recommended for administration in the evening hours. Moreover, alendronate (FOSAMAX® manufactured by Merck & Co.) is known to have an extremely low bioavailability following oral administration. Alendronate (FOSAMAX® manufactured by Merck & Co.) is recommended to be taken on an empty stomach upon waking in the morning. It is also recommended that the patient stay in an upright position for at least 30 minutes following administration. Therefore the presently preferred embodiment of the present invention is ideally suited for increasing patient compliance.

Thus, by varying the composition of the secondary capsule wall, release of the contents of the secondary capsule may be targeted to an optimal delivery time. A differential release between the primary capsule and secondary capsule may therefore optimize management of co-morbid conditions in a patient. The patient is able to take a single daily administration vehicle, which provides a plurality of medicaments for managing co-morbid conditions. Thus, the incorporation of time-release coatings in the encapsulation process when forming a multi-compartment capsule may be specifically designed to fit the needs and desires of numerous different users having similar conditions that are being targeted for treatment. It is intended, therefore, that the examples provided herein be viewed as exemplary of the principles of the present invention, and not as restrictive to a particular structure or method for implementing those principles.

### Example IV

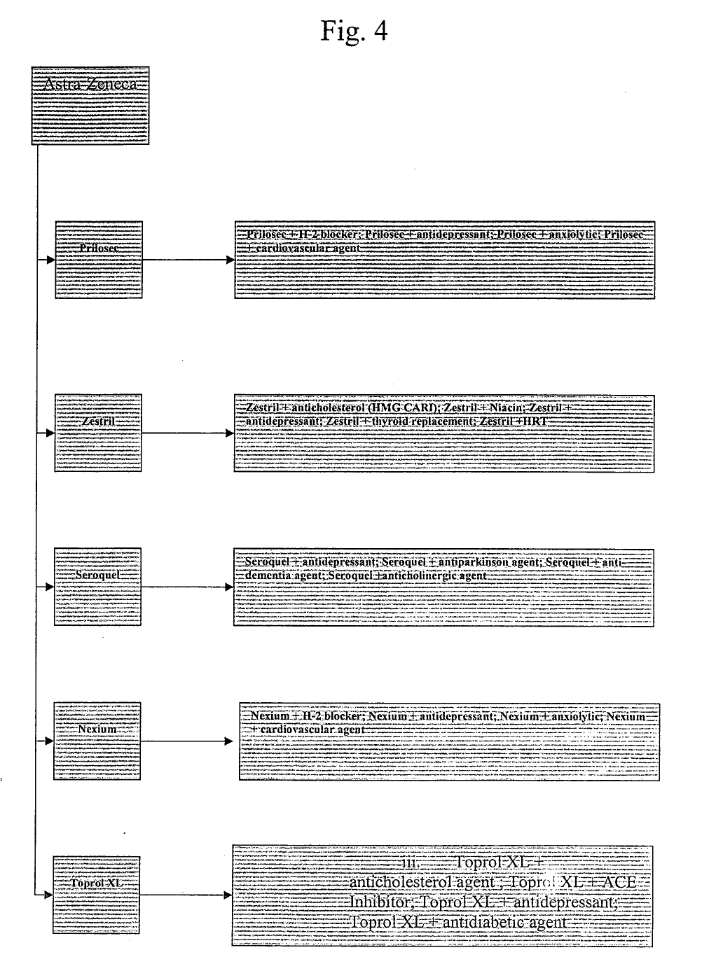

Referring to FIG. 4, in one presently preferred embodiment of the present invention, a plurality of medicaments are delivered in a single vehicle for management of co-morbid diseases, illnesses or conditions. More particularly, a plurality of medicaments comprising a beta-adrenergic receptor antagonist, for example, metoprolol (TOPROL XL® manufactured by AstraZeneca) and a proton pump inhibitor, for example, omeprazole (PRILOSEC® manufactured by AstraZeneca) may be combined into a single delivery apparatus, for example, a multi-compartment capsule.

In the presently preferred embodiment, a therapeutically effective amount of omeprazole (PRILOSEC® manufactured by AstraZeneca) may be introduced into a receiving chamber in a secondary capsule. The base and cap of the secondary capsule may then be sealed using any available conventional means readily known to those skilled in the art. The secondary capsule may then be introduced into a receiving chamber in a primary capsule. A therapeutically effective amount of metoprolol (TOPROL XL® manufactured by AstraZeneca) may be introduced into a receiving chamber in a primary capsule. The base and cap of the primary capsule may then be sealed using any available conventional means readily known to those skilled in the art.

A capsular format of the present invention may include the following composition:

Primary Capsule:

| | |
|---|---|
| metoprolol (TOPROL XL ®manufactured by AstraZeneca) | 100 mg |
| [dosage range 25-200 mg/day] | |

Secondary Capsule:

| | |
|---|---|
| omeprazole (PRILOSEC ® manufactured by AstraZeneca) | 20 mg |
| [dosage range 20-80 mg/day] | |

The incorporation of time-release coatings to varying the release rates of the medicaments (e.g., metoprolol (TOPROL XL® manufactured by AstraZeneca) and omeprazole (PRILOSEC® manufactured by AstraZeneca)) in the primary and secondary capsules, respectively, of the multi-compartment capsule may be used to target key time intervals or events when the body may be most able to utilize the named active ingredients. For example, the peak acid secretion in the gastrointestinal system is believed to occur during sleep. Proton pump inhibitors may therefore be more efficacious when administered in the evening hours and thus allowing a peak systemic concentration to coincide with higher levels of acid production. Therefore, to maximize its effects, omeprazole (PRILOSEC® manufactured by AstraZeneca) is recommended for administration in the evening hours. Therefore the presently preferred embodiment of the present invention is ideally suited for increasing patient compliance.

Thus, by varying the composition of the secondary capsule wall, release of the contents of the secondary capsule may be targeted to an optimal delivery time. A differential release between the primary capsule and secondary capsule may therefore optimize management of co-morbid conditions in a patient. The patient is able to take a single daily administration vehicle, which provides a plurality of medicaments for managing co-morbid conditions. Thus, the incorporation of time-release coatings in the encapsulation process when forming a multi-compartment capsule may be specifically designed to fit the needs and desires of numerous different users having similar conditions that are being targeted for treatment. It is intended, therefore, that the examples provided herein be viewed as exemplary of the principles of the present invention, and not as restrictive to a particular structure or method for implementing those principles.

### Example V

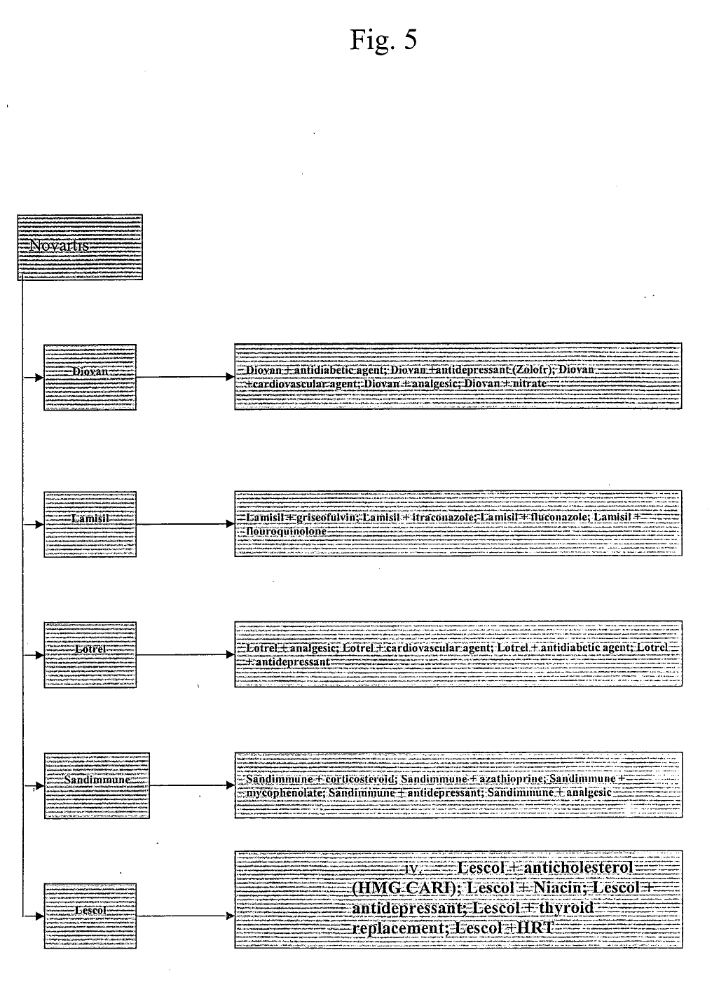

Referring to FIG. 5, in one presently preferred embodiment of the present invention, a plurality of medicaments are delivered in a single vehicle for management of co-morbid diseases, illnesses or conditions. More particularly, a plurality of medicaments comprising an acetylcholinesterase inhibitor, for example, rivastigmine (EXELON® manufactured by Novartis, A.G) and an angiotensin II receptor antagonist, for example, valasartan (DIOVAN®manufactured by Novartis, A.G) may be combined into a single delivery apparatus, for example, a multi-compartment capsule.

In the presently preferred embodiment, a therapeutically effective amount of valasartan (DIOVAN® manufactured by Novartis, A.G) may be introduced into a receiving chamber in a secondary capsule. The base and cap of the secondary capsule may then be sealed using any available conventional means readily known to those skilled in the art. The secondary capsule may then be introduced into a receiving chamber in a primary capsule. A therapeutically effective amount of rivastigmine (EXELON® manufactured by Novartis, A.G) may be introduced into a receiving chamber in a primary capsule. The base and cap primary capsule may then be sealed using any available conventional means readily known to those skilled in the art.

A capsular format of the present invention may include the following composition:

Primary Capsule:

| | |
|---|---|
| rivastigmine (EXELON ® manufactured by Novartis, A.G) | 9 mg |
| [dosage range 6-12 mg/day] | |

Secondary Capsule:

| | |
|---|---|
| valasartan (DIOVAN ®manufactured by Novartis, A.G) | 120 mg |
| [dosage range 80-320 mg/day] | |

The incorporation of time-release coatings to varying the release rates of the medicaments (e.g., rivastigmine (EXELON® manufactured by Novartis, A.G) and valasartan (DIOVAN® manufactured by Novartis, A.G)) in the primary and secondary capsules, respectively, of the multi-compartment capsule may be used to target key time intervals and optimize patient compliance. For example, rivastigmine (EXELON® manufactured by Novartis, A.G) is labeled for use in patients suffering from Alzheimer's disease, a type of dementia. These patients often have difficulty remembering tasks associated with activities of daily living. It is known that these patients may forget to take their medicines. By reducing the daily administration of medicine in this patient, there may be improved patient compliance. Therefore the presently preferred embodiment of the present invention is ideally suited for increasing patient compliance.

Thus, by varying the composition of the secondary capsule wall, release of the contents of the secondary capsule may be targeted to an optimal time. A differential release between the primary capsule and secondary capsule may therefore optimize management of co-morbid conditions in a patient. The patient is able to take a single daily administration vehicle, which provides a plurality of medicaments for managing co-morbid conditions. Thus, the incorporation of time-release coatings in the encapsulation process when forming a multi-compartment capsule may be specifically designed to fit the needs and desires of numerous different users having similar conditions that are being targeted for treatment. It is intended, therefore, that the examples provided herein be viewed as exemplary of the principles of the present invention, and not as restrictive to a particular structure or method for implementing those principles.

As can be seen above, various embodiments of the present invention can be utilized in specific medical applications. By way of example only and not by way of limitation, the present invention can be practiced to prepare delivery devices for use in chemotherapy to address/treat, by way of example and not by limitation, the following aspects of chemotherapy: psychological, timing (to coincide with tumor growth for example) route of administration, nausea, vomiting (CINV), compliance, and cost (e.g. reduce hospital management of patients, reduce the number of “repeat” drug doses due to patient vomiting, etc.). Still further, the just mentioned aspects are not limited to chemotherapy, as the present invention can be practiced to address common aspects between chemotherapy and other treatments.

Further by way of examples, capsules containing Zofran (ondansertron), Temodar (temozolomide) can be made.

Still further, the present invention can be used in cardiovascular treatments, for example hypertension, heart failure, and heart rhythm disorders. Also, the present invention can be used in immunology (e.g. transplant rejections, auto-immune disorders, etc.). The present invention can be used to treat neurological disorders (such as Parkinson's disease, dementia, stroke, epilepsy, and migraine headache, etc.), psychiatric disorders (schizophrenia, bipolar disease, depression, anxiety, ADHD/ADD, Addictions, etc.), infectious diseases (fungal, bacterial, viral (HIV), etc.), and in anesthesiology (induction anesthesia, local anesthesia). Furthermore, the present invention has application in endocrinology (cholesterol, diabetes, hormone replacement therapy, thyroid dysfunction, oral contraception, obesity, etc.), dermatology (onychomycosis, acne, rosaceae, psoriasis, etc.), rheumatology (arthritis, gout, osteoporosis/Osteomalacia), respiratory fields (asthma, emphysema, cystic fibrosis, etc.), gastro-intestinal fields (gastro-esophageal reflux disease, ulcer prophylaxis, crohn's disease, inflammatory bowel disease, etc.), chronic renal failure (vitamin and mineral replacement, blood pressure regulation, diabetes, depression, etc.), genito-urinary (enlarged prostate/BPH, overactive bladder, erectile dysfunction, feminine yeast infections, etc.) and hematology-oncology (thromboembolous, hermatopoeisis, neoplastic disease, nausea/vomiting).

The present invention may be utilized for dual/multiple disease state therapy with one capsule, dual/multiple organ system therapy with one capsule, enhanced pharmacoeconomic delivery, temporal/circadian optimized delivery, sports nutrition, sports therapy, athletic performance enhancement and law enforcement and military applications.

The present invention may be embodied in other specific forms without departing from its spirit or essential characteristics. The described embodiments are to be considered in all respects only as illustrative, and not restrictive. One skilled in the art will recognize that this technology may be applicable for providing a wide variety a plurality of medicaments in a single delivery vehicle for management of co-morbid diseases, illnesses or conditions.

Other drugs that may be combined, into a single delivery apparatus, for example, a multi-compartment capsule, according to the present invention include, by way of example, Lipitor (by Pfizer), Prilosec (by Astra Zenica), Prevacid (by Tap), Zocor (by Merck), Celebrex (by Searle), Zoloft (by Pfizer), Paxil (by GSK), Vioxx (by Merck), Prozac (by Lilly), Augmentin (by GSK), Norvasc (by Pfizer), Zyprexa (by Lilly) EPO (by Amgen), Cipro (by Bayer), Lotrel (by Novartis), Topamx (by Johnson and Johnson), Glucotrol XL (by Pfizer), Claritin (by Schwiring), Wellbutrin SR (by GSK), Difucan (by Pfizer), Prilosec/Nexium (by Astra Zenica), Accupril (by Pfizer), Zofran (by GSK), Zithromax (by Pfizer), Imitrex (by GSK), Fosamax (by Merck) Zytrec (by Pfizer), Neurontin (by Pfizer), Pravachol (by BMS), Depakote (by Abbott). Some embodiments of the invention include two or more of the just mentioned drugs combined in a single delivery apparatus, while other embodiments of the invention include only one of the just mentioned drugs combined with another pharmaceutical and/or nutriceutical and/or other substances, such as, by way of example and not by way of limitation, one or more of the pharmaceuticals and/or nutraceuticals and/or other substances listed in Appendix I. Still further, some embodiments of the invention include two or more of the pharmaceuticals and/or nutraceuticals and/or other substances listed in Appendix I in a single delivery apparatus. In this regard, the present invention includes an apparatus and a method for delivering a plurality of the medicaments listed in Appendix I for management of co-morbid diseases, illnesses, and/or conditions, as would be understood by one of ordinary skill in the art furnished with the teachings of the present invention.

It is further noted that a singe delivery apparatus, to deliver a plurality of medicaments for management of co-morbid diseases, illnesses or conditions according to the present invention may include a deliver apparatus as is disclosed in U.S. application Ser. No. 10/369,427, filed Feb. 18, 2003, entitled “MULTI-PHASE, MULTI-COMPARTMENT CAPSULAR DELIVERY APPARATUS AND METHODS FOR USING SAME,” the contents of which are hereby incorporated by reference in their entirety; U.S. application Ser. No. 10/368,951, filed Feb. 18, 2003, entitled “PROCESS FOR ENCAPSULATING MULTI-PHASE, MULTI-COMPARTMENT CAPSULES,” the contents of which are hereby incorporated by reference in their entirety; U.S. application Ser. No. 10/369,244, filed on Feb. 18, 2003, and entitled “MULTI-PHASE, MULTI-COMPARTMENT CAPSULAR DELIVERY APPARATUS FOR THERAPEUTIC COMPOSITIONS AND METHODS FOR USING SAME” the contents of which are hereby incorporated by reference in their entirety; U.S. application Ser. No. 10/369,247, filed Feb. 18, 2003, and entitled “PROCESS FOR ENCAPSULATING MULTI-PHASE, MULTI-COMPARTMENT CAPSULES FOR THERAPEUTIC COMPOSITIONS,” the contents of which are hereby incorporated by reference in their entirety; and PCT/US/03/10816 filed Apr. 9, 2003, and entitled “MULTI-PHASE, MULTI-COMPARTMENT CAPSULAR SYSTEM”, the contents of which are hereby incorporated by reference in their entirety, the apparatus delivering these pharmaceuticals and/or nutraceuticals and/or other substances containing them in a common phase (e.g., all solid, all liquid, etc.), or in a multi-phase (e.g., one substance solid and one substance liquid; two substances solid and one substance liquid, etc.) state as disclosed in the just mentioned references.

### APPENDIX I

### Medicaments Acting on the Autonomic Nervous System

Adrenergic Medicaments

Cholinergic Medicaments

Direct Muscarinic Agonists Choline Esters
acetylcholine
bethanechol (Urecholine)
carbachol
methacholine (Provocholine)

Alkaloids
muscarine
pilocarpine (Pilocar)

Direct Nicotinic Agonist
nicotine

Acetylcholinesterase Inhibitors Acetylcholinesterase Inhibitors (“Reversible”)
edrophonium (Tensilon)
neostigmine (Prostigmin)
physostigmine (Antilirium)

Acetylcholinesterase Inhibitors (“Irreversible”)
(diisopropylflurorphosphate DFP)
echothiophate (Phospholine)
isoflurophate (Floropryl)

Muscarinic Antagonists Atropine
ipratropium (Atrovent)
pirenzepine
scopolamine

2-PAM: Acetylcholinesterase Reactivator Pralidoxime (Protopam) {2-PAM}:peripheral acetylcholinesterase reactivator for certain phosphoryl-enzyme complexes

Ganglionic Blockers
hexamethonium
mecamylamine (Inversine)
trimethaphan

Catecholamines
dobutamine (Dobutrex)
dopamine (Intropin)
epinephrine
isoproterenol (Isuprel)
norepinephrine (Levophed)

Direct Adrenoceptor Agonist Medicaments
albuterol (Ventolin, Proventil)
clonidine (Catapres)
methoxamine (Vasoxyl)
oxymetazoline (Afrin)
phenylephrine (Neo-Synephrine)
ritodrine (Yutopar)
salmeterol (Serevent)
terbutaline (Brethine)

Indirect-Acting Sympathomimetic Medicaments
amphetamine
cocaine
ephedrine, Pseudoephedrine
tyramine

Alpha-Adrenoceptor Antagonists Medicaments
doxazosin (Cardura)
labetalol (Trandate, Normodyne)
phenoxybenzamine (Dibenzyline)
phentolamine (Regitine)
prazosin (Minipress)
terazosin (Hytrin)
tolazoline (Priscoline)
trimazosin
yohimbine (Yocon)

β-Adrenoceptor antagonist Medicaments
atenolol (Tenormin)
butoxamine
esmolol (Brevibloc)
labetalol (Trandate, Normodyne)
metoprolol (Lopressor)
nadolol (Corgard)
pindolol (Visken)
propranolol (Inderal)
timolol (Blocadren)

Adrenergic Neuron Blocking Medicaments
guanethidine (Ismelin)
reserpine

### Cardiovascular System Disorders Cardiovascular Testing and Diagnosis

Hypertension (HTN)

Heart Failure

Ischemic Heart Disease

Myocardial Infarction

Arrhythmias

Isolated Diastolic Heart Failure and Cardiomyopathies

Cardiac Transplantation

Venous Thromboembolism

Stroke

Hyperlipidemia

Peripheral vascular disease

Diuretics
carbonic-anhydrase inhibitors
loop diuretics
osmotic diuretics
potassium sparing diuretics
thiazide diuretics

### Anitiarrhythmic Medicaments

Sodium Channel blocking agents
disopyramide (Norpace)
flecainide (Tambocor)
ibutilide
lidocaine (Xylocalne)
mexiletine (Mexitil)
moricizine (Ethmozine)
procainamide (Pronestyl, Procan)
propafenone (Rythmol)
quinidine
tocainide (Tonocard)

Calcium Channel blocking agents
bepridil (Vasocor)
diltiazem (Cardizem)
verapamil (Isoptin, Calan)

Adrenergic receptor antagonists
propranolol (Inderal)

Other medicaments
adenosine (Adenocard)
amiodarone (Cordarone)
bretylium (Bretylol)
disopyramide (Norpace)
esmolol (Brevibloc)
sotalol (Betapace)

### Hypolipidemic Medicaments

HMG CoA Reductase Inhibitors
atorvastatin (Lipitor)
cerivistatin (Baycol)
lovastatin (Mevacor)
pravastatin (Pravochol)
simvastatin (Zocor)

Bile-acid sequestrants
cholestyramine (Questran)
colestipol (Colestid)

Fibric acids
clofibrate
fenofibrate (Tricor)
gemfibrozil (Lopid)

niacin, nicotinic acid

probucol (Lorelco)

### Antihypertensive Medicaments

Adrenergic receptor antagonists
acebutalol (Sectral)
atenolol (Tenormin)
betaxolol (Betoptic)
bisoprolol (Zebeta)
carteolol (Cartrol)
clonidine (Catapres)
labetalol (Normodyne)
metoprolol (Toprol)
penbutalol (Levatol)
pindolol (Visken)
prazosin (Minipres)
propranolol (Inderal)
terazosin (Hytrin)
timolol (Timoptic)

Calcium Channel Antagonists
amlodipine (Norvasc)
diltiazem (Cardizem)
felodipine (Plendil)
isradipine (Dynacirc)
nicardipine (Cardene)
nifedipine (Procardia)
nimodipine (Nimotop)
nisoldipine (Sular)
verapamil (Isoptin, Calan)

Angiotensin Converting Enzyme (ACE) Inhibitor
benazepril (Lotensin)
bepridil (Vascor)
captopril (Capoten)
enalapril (Vasotec)
fosinopril (Monopril)
lisinopril (Prinivil, Zestril)
moexipril (Univasc)
quinapril (Accupril)
ramipril (Altace)

Angiotensin II Receptor Antagonists
losartan (Cozaar)
valasartan (Diovan)

Diuretics
amiloride (Midamor)
bumetanide (Bumex)
chlorothalidone (Hygroton)
ethacrynic acid (Edecrin)
furosemide (Lasix)
hydrochlorothiazide (Diuril)
indapamide (Lozol)
metolazone (Zaroxolyn)
torsemide (Demadex)
triamterene

Other Agents
hydralazine (Apresoline)
minoxidil (Rogaine)
nitroprusside (Nipride)
prazosin (Minipres)
reserpine
sotalol (Brevibloc)
spironolactone (Aldactone)
terazosin (Hytrin)

### Antianginal Medicaments

Organic nitrates

Calcium Channel Antagonists

Adrenergic Receptor Antagonists

amyl nitrite

erythrityl tetranitrate

isosorbide dinitrate (Isordil)

nitroglycerin

pentaerythritol tetranitrate

### Congestive Heart Failure Medicaments

phosphodiesterase (PDE) inhibitors
amrinone (Inocor)
milrinone (Primacor)

carvedilol (Coreg)

cardiac glycosides
digitoxin
digoxin

diuretics

ACE Inhibitors

dobutamine dopamine

### Respiratory System Disorders Asthma

Chronic Obstructive Lung Disease (COLD)/Chronic Obstructive Pulmonary Disease (COPD)

Acute Respiratory Distress Syndrome (ARDS)

Drug-Induced Pulmonary Disease

Cystic Fibrosis

corticosteroids
beclomethasone
betamethasone
cortisone
dexamethasone
fluticasone (Flovent/Flonase)
hydrocortisone
methylprednisolone
prednisolone
prednisone
triamcinolone

sympathomimetics
albuterol (Proventil/Ventolin)
salmeterol (Serevent)

muscarinic antagonists
ipratropium (Combivent)

leukotriene pathway inhibitors
montelukast (Singulair)
zafirlukast (Accolate)

mast cell stabilizers
cromolyn (Intal)

methylxanthines
theophylline
aminophylline

Dnase (Pulmozyme)

### Gastrointestinal System Disorders Gastro-Esophageal Reflux Disease (GERD)

Peptic Ulcer Disease

Inflammatory Bowel Disease

Nausea and Vomiting

Diarrhea, Constipation, Irritable Bowel Disease (IBD)

Portal Hypertension and Cirrhosis

Drug-Induced Liver Disease

Pancreatitis

Viral Hepatitis

Liver Transplantation

Histamine-2 receptor antagonists
famotidine (Pepcid)
nizatidine (Axid)
pantoprazole (Protonix)
rabeprazole (Aciphex)
ranitidine (Zantac)

Proton Pump Inhibitors (PPIs)
esomeprazole (Nexium)
lansoprazole (Prevacid)
omeprazole (Prilosec)

Anti-nausea/anti-vertigo medicaments
anticholinergics
   antihistamines (Histamine-1 receptor antagonists)
dopamine antagonists
prokinetic gastric stimulant
serotonin 5HT₃ receptor antagonists
   dolasetron (Anzmet)
   granisetron (Kytril)
   ondansetron (Zofran)
other medicaments
   hydroxyzine (Atarax, Vistaril)
   corticosteroids
   benzodiazepines
   cannabinoids

Prokinetic gastric stimulants (gastric motility stimulants)
cisapride (Propulsid))
metoclopramide (Reglan)

Laxatives
Saline laxatives
   magnesium salts
   sodium salts
irritant/stimulant medicaments
   cascara
   senna
   phenolphthalein
   bisacodyl
   casanthran of
   castor oil
bulk-producing medicaments
   methylcellulose
   psyllium
   polycarbophil
lubricant
   mineral oil
surfactants
   docusate
miscellaneous
   glycerin
   lactulose

Anti-diarrheal medicaments
diphenoxylate
atropine
diphenoxin
loperamide
bismuth
lactobacillus

Ulcerative Colitis Medicaments
mesalamine
olsalazine

### Renal System Disorders Acute Renal Failure

Progressive Renal Failure/Chronic Renal Failure

### Neurologic System Disorders Multiple Sclerosis and Inflammatory Polyneuropathies

Epilepsy

Parkinson's disease and Movement Disorders

Pain management

Headache

Amyotrophic Lateral Sclerosis

Anti-Epileptic Medicaments
carbamazepine (Tegretol)
divalproex sodium (Depakote)
felbamate (Felbatol)
gabapentin (Neurontin)
lamotrigine (Lamictal)
oxcarbazepine (Trileptal)
phenytoin (Dilantin)
topiramate (Topamax)
zonisamide (Zonegran)

Antimigraine Medicaments
serotonin 5HT_{1d} receptor agonists
   almotriptan (Axert)
   frovatriptan (Frova)
   naratriptan (Amerge)
   rizatriptan (Rizalt)
   sumatriptan (Imitrex)
   zolmitriptan (Zomig)
ergot alkaloids
   dihydroergotamine (DHE)
isometheptine/dichlorophenazone (Midrin)
caffeine
pizotifen (Sanomigran)

Sedative-Hypnotic Medicaments
benzodiazepines
   alprazolam (Xanax)
   clonazepam (Klonopin)
   clorazepate (Tranxene)
   diazepam (Valium)
   flumazenil (Romazicon)—antagonist
   lorazepam (Ativan)
   midazolam (Versed)
   triazolam (Halcion)
barbiturates/Anesthetics
   pentobarbital (Nembutal)
   phenobarbital (Luminal)
   thiopental (Pentothal)
non-depressant anxiolytic
   buspirone (BuSpar)

Treatment of Alcoholism
disulfiram (Antabuse)

Pain Management Medicaments
Opioids
   Opioid Peptides
      beta-endorphin
      dynorphin
         enkephalins
   Agonists
      codeine
      etorphine
      fentanyl (Sublimaze)
      hydrocodeine
      hydromorphone
      meperidine (Demerol)
      methadone (Dolophine)
      morphine
      oxycodone
      propoxyphene
   Agonist-antagonists
      buprenorphine
   Partial Agonist
      dezocine (Dalgan)
      nalbuphine (Nubain)
      pentazocine (Talwain)
   Antagonist
      naloxone (Narcan)
Non-opiate
   acetaminophen (tylenol)
   tramadol (ultram)

Anti-Parkinsonism Medicaments
levodopa
carbidopa
bromocriptine (Parlodel)
pergolide (Permax)
amantadine (Symmetrel)
selegiline (Deprenyl)
anticholinergic agents
dopamine Agonists
   pramipexole (Mirapex)
   ropinirole (Requip)
COMT inhibitors
   entacapone (Comtan)
   tolcapone (Tasmar)

Anti-Spasticity Medicaments
baclofen (Lioresal)
botulinum toxin type A (Botox)
carisoprodol (Soma, Rela)
chlorphenesin (Maolate)
chlorzoxazone (Paraflex)
cyclobenzaprine (Flexeril)
dantrolene (Dantrium)
diazepam (Valium)
metaxalone (Skelaxin)
methocarbamol (Robaxin)
orphenadrine (Norflex)
tizanidine (Zanaflex)

### Psychiatric System Disorders Childhood Psychiatric Disorders

Attention Deficit Hyperactivity Disorder (ADHD)/Attention Deficit Disorder (ADD)

Eating disorders

Alzheimer's disease and Dementia Disorders

Substance abuse and Addictive Disorders
alcohol, tobacco and caffeine abuse

Schizophrenia

Depressive disorders

Bipolar disorders

Anxiety disorders

Obsessive-Compulsive disorders

Sleep disorders

Psychostimulant Medicaments
amphetamine mixed salts (Adderall)
dextroamphetamine (Dexedrine)
methylphenidate (Ritalin, Concerta)

Antipsychotic Medicaments (dopamine antagonists)
Phenothiazine type
   chlorpromazine (Thorazine)
   fluphenazine (Prolixin)
Thioxanthene type
   thiothixene (Navane)
Butyrophenone type
   haloperidol (Haldol)
Dibenzodiazepine type
   clozapine (Clozaril)
Thienobenzodiazepine type
   olanzapine (Zyprexa)
   quetiapine (Seroquel)

Antidepressant Medicaments
Tricyclic antidepressants (TCA's)
   amitriptyline (Elavil, Endep)
   clomipramine (Anafranil), also a SSRI
   desipramine (Norpramin)
   doxepin (Sinequan)
   imipramine (Tofranil)
   maprotiline (Ludiomil)
   nortriptyline (Aventyl, Pamelor)
   protriptyline (Vivactil)
Monoamine oxidase inhibitors (MAO-I's)
   clorgyline (specific for MAO type A)
   isocarboxazid (Marplan)
   phenelzine (Nardil)
   tranylcypromine (Parnate)
Second Generation Medicaments (not including SSRIs)
   amoxapine (Asendin)
   bupropion (Wellbutrin)
   nefazodone (Serzone)
   trazodone (Desyrel)
Serotonin-Specific Reuptake Inhibitors (SSRIs)
   citalopram (Celexa)
   clomipramine (Anafranil)
   escitalopram (Lexapro)
   fluoxetine (Prozac)
   fluvoxamine (Luvox)
   paroxetine (Paxil)
   sertraline (Zoloft)
Other
   lithium
   mirtazapine (Temeron)
   venlafaxine (Effexor)

Anti-Anxiety Agents
barbiturates
benzodiazepines
buspirone (Buspar)
chloral hydrate
doxepin
hydroxyzine
sedative-hypnotics
serotonin reuptake inhibitors

Anti-Demential Medicaments
cholinesterase inhibitors
   donepezil (Aricept)
   galantamine (Reminyl)
   rivastigmine (Exelon)
   tacrine (Cognex)

### Endocrinologic System Disorders Diabetes Mellitus

Thyroid disorders

Adrenal Gland disorders

Pituitary Gland disorders

ACTH

Adrenal androgens

Adrenocortical Function Antagonists

Mineralocorticoid antagonists

Anti-Diabetic Medicaments
Insulin
Sulfonylureas
   acetohexamide (Dymelor)
   chlorpropamide (Diabinese)
   glimepiride (Amaryl)
   glipizide (Glucotrol)
   glyburide (Micronase, DiaBeta)
   tolazamide (Tolinase)
   tolbutamide (Orinase)
Biguanides
   metformin (Glucophage)
Alpha-glucosidase Inhibitors
   acarbose (Precose)
   miglitol (Glyset)
Thiazolidinedione Derivatives
   pioglitazone (Actos)
   rosiglitazone (Avandia)
   troglitazone (Rezulin)

Thyroid Disorder Medicaments
Levothyroxine
Liothyronine
Liotrix

Hypothalamic and Pituitary Gland Medicaments
bromocriptine (Parlodel)
chorionic gonadotropin (hCG)
corticotropin generic (ACTH)
cosyntropin (Cortrosyn)
desmopressin (DDAVP)
gonadorelin acetate (GnRH) (Lutrepulse)
gonadorelin hydrochloride (GnRH) (Factrel)
goserelin acetate (Zoladex)
growth hormone
histrelin (Supprelin)
leuprolide (Lupron)
menotropins (hMG) (Pergonal, Humegon)
nafarelin (Synarel)
octreotide (Sandostatin)
oxytocin (Pitocinit, Syntocinon)
pergolide (Permax)
protirelin (Thypinone, Relefact TRH)
sermorelin (GHRH) (Geref)
somatrem (Protropin)
somatropin (Humatrope, Nutropin)
thyrotropin (TSH) (Thytropar)
urofollitropin (Metrodin)
vasopressin (Pitressin Synthetic)

### Gynecologic System and Obstetric Conditions Pregnancy and Lactation

Infertility

Contraception

Menstruation-related disorders

Endometriosis

Hormone Replacement Therapy (HRT)

Conjugated estrogens (Premarin)

desogestrel

di-norgestrel

ethinyl diacetate

ethinyl estradiol

levonorgestrel

medroxyprogesterone

norethindrone

norgestimate

progesterone

### Urologic System Disorders Erectile Dysfunction

Benign Prostatic Hypertrophy

Urinary Incontinence

apomorphine

alprostadil

phosphodiesterase (PDE-5) inhibitors
sildenafil (Viagra)
tadalafil (Cialis)
vardenafil (Levitra)

tolterodine (Detrol)

tamulosin (Flomax)

yohimbine

### Immunologic System Disorders Systemic Lupus Erythematosus and Other Collagen-vascular Diseases

Allergic and pseudo-allergic drug reactions

### Bone and Joint System Disorders Osteoporosis and Osteomalacia

Rheumatoid Arthritis

Osteoarthritis

Gout and hyperuricemia

Medicaments Used in the Control of Inflammation
Non-steroidal anti-inflammatory drugs (NSAIDs)
   aspirin
   diclofenac (Cataflam, Voltaren)
   diflusnisal (Dolobid)
   etodolac (Lodine)
   fenoprofen (Nalfon)
   flubiprofen (Ansaid)
   ibuprofen (Motrin, Advil, Nuprin)
   indomethacin (Indocin)
   ketoprofen (Orudis)
   ketorolac (Toradol)
   meclofenamate
   nabumetone (Relafen)
   naproxen (Naprosyn)
   oxaprozin (Daypro)
   phenylbutazone
   piroxicam (Feldene)
   salicylate
   sulindac (Clinoril)
   tolmetin (Tolectin)
Cyclooxygenase-2 inhibitors (COX-2)
   celecoxib (Celebrex)
   rofecoxib (Vioxx)

Arthritis and Gout Medicaments
allopurinol
anti-malarial compounds
   chloroquine
colchicine
enbrel
Glucocorticoids
Gold
methotrexate
NSAIDs
Penicillamine

Other Medicaments
alendronate (Fosamax)
raloxifene (Evista)

### Disorders of the Eyes, Ears, Nose, and Throat Systems Glaucoma

Allergic rhinitis
Histamine-1 receptor antagonists
   brompheniramine (Dimetane)
   cetirizine (Zyrtec)
   chlorpheniramine (Chlor-Trimeton)
   clemastine (Tavist)
   cyproheptadine (Periactin)
   dimenhydrinate (Dramamine)
   diphenhydramine (Bendaryl)
   doxylamine (Sominex, Unisom)
   fexofenadine (Allegra)
   loratidine (Claritin)
Sympathomimetic medicaments
   pseudoephedrine (Sudafed)

### Dermatologic System Disorders Acne

Psoriasis

Rosacea and pigmentation disorders

Drug-Induced skin reactions

### Hematologic System Disorders Hematopoeisis

Anemias

Coagulation disorders

Sickle-cell anemia

Drug-induced hematologic disorders

Coagulation disorders Medicaments
aspirin
clopidogrel (Plavix)
fibrinolytic inhibitors
fibrinolytics
glycoprotein (GP) IIb/IIIa antagonists/monoclonal antibodies
   abciximab (Reopro)
   eptifibatide (Integrelin)
   tiofibran (Aggrastat)
heparin
low-molecular weight heparins
Plasma fractions—blood factors
ticlopidine (Ticlid)
vitamin K
warfarin (Coumadin)

### Infectious System Diseases Central Nervous System (CNS) Infections

Lower Respiratory Tract Infections

Upper Respiratory Tract Infections

Skin and Soft Tissue Infections

Infective Endocarditis

Tuberculosis

Gastrointestinal Infections and Enterotoxigenic poisonings

Intra-abdominal Infections

Parasitic diseases

Urinary Tract Infections and Prostatis

Sexually Transmitted Diseases

Bone and Joint Infections

Sepsis and Septic Shock

Superficial Fungal Infections

Invasive Fungal Infections

Infections in Immunocompromised Patients

Antimicrobial prophylaxis in Surgery

Vaccines, toxoids, and other immunobiologics

Human Immunodeficiency Virus Infection

Medicaments Used in Infectious Diseases

Cell Wall Synthesis Inhibitors
Penicillins
   amoxicillin (Amoxil Polymox)
   ampicillin (Principen, Omnipen)
   benzathine Penicillin G
   benzyl Penicillin (Penicillin G)
   carbenicillin (Geocillin)
   cloxacillin (Cloxapen)
   dicloxacillin (Dynapen)
   methicillin (Staphcillin)
   mezlocillin
   nafcillin (Nafcil, Unipen)
   oxacillin
   phenoxymethyl Penicillin (Penicillin V)
   piperacillin (Pipracil)
   ticarcillin (Ticar)
Cephalosporins
   1st generation:
      cefazolin (Ancef, Defzol)
      cephalexin (Keflex)
      cephalothin (Keflin)
   2nd generation:
      cefaclor (Ceclor)
      cefoxitin (Mefoxin)
      cefpodoxime (Vantin)
      cefuroxime (Zinacef, Ceftin)
      loracarbef (Lorabid)
   3rd generation:
      cefoperazone
      cefotaxime (Claforan)
      cefotetan
      ceftazidime (Fortax, Taxidime, Tazicef)
      ceftriaxone (Rocephin)
      veftizoxime (Cefizox)
   4th generation:
      cefepime
Other beta-Lactams aztreonam (Azactan)
   clavulanic acid
   imipenem (Primaxin)
   meropenem (Merrem IV)
   sulbactam
Other Cell-Wall Synthesis Inhibitors
   bacitracin
   cycloserine
   fosfomycin (Monurol)
   vancomycin (Vancocin)

Agents Which Affect Cell Membranes
Polymixins
   Colistim ethate
   Polymyxin B

Protein Synthesis Inhibitors
Aminoglycosides
   amikacin (Amikin)
   gentamicin (Garamycin)
   kanamycin (Kantrex)
   neomycin
   netilmicin (Netromycin)
   streptomycin
   tobramycin
Tetracyclines
   demeclocycline (Declomycin)
   doxycycline
   doxycycline (Vibramycin, Doryx)
   tetracycline (Achromycin)
Macrolides
   azithromycin (Zithromax)
   clarithromycin (Biaxin)
   erythromycin esters
   erythromycin
Other Protein Synthesis Inhibitors
   Chloramphenicol (Chloromycetin)
   Clindamycin (Cleocin)
   Spectinomycin (Trobicin)

Inhibitors of Folate-Dependent Pathways
co-trimoxazole
silver Sulfadiazine
sodium Sulfacetamide
sulfamethoxazole (Gantanol)
sulfasalazine (Azulfidine) (Salicylazosulfapyridine)
sulfisoxazole (Gantrisin)
sulfonamides

Dihydrofolate Reductase Inhibitor
trimethoprim

DNA Gyrase Inhibitors
ciprofloxacin (Cipro)
gatifloxacin (Tequin)
levofloxacin (Levaquin)
lomefloxacin (Maxaquin)
nalidixic acid
ofloxacin (Floxin)

Urinary Tract Antiseptics
nitrofurantoin

Antimyobacterial Agents
First-line anti-TB medicaments
   ethambutol
   isoniazid (INH)
   pyrazinamide
   rifampin (Rimactane)
   streptomycin
Second-line anti-TB medicaments
   capreomycinA
   cycloserine
   dapsone
   ethionamide
   para-aminosalicylic acid

AntiFungal Agents
amphotericin B (Fungizone, Amphotec)
clotrimazole (Mycelex)
fluconazole (Diflucan)
flucytosine
griseofulvin
itraconazole (Sporanox)
ketoconazole (Nizoral)
miconazole (Monistat)
nystatin (Mycostatin)

AntiParasitic Agents

Antimalarials
chloroquine (Aralen)
mefloquine (Lariam)
primaquine
pyrimethamine-sulfadoxine (Fansidar)

Antiprotozoals
metronidazole (Flagyl)
pentamidine isethionate
pyrimethamine-sulfonamide
trimethoprim (generic) sulfamethoxazole (Gantanol)

Antihelminthic Medicaments
mebendazole
praziquantel (Biltricide)
pyrantel pamoate
thiabendazole (Mintezol)

Antiviral Medicaments
acyclovir (Zovirax)
didanosine (DDI)
foscarnet (Foscavir)
ganciclovir (DHPG, Cytovene)
ribavirin
rimantadine
stavudine (d4T))
valacyclovir (Valtrex)
vidarabine (Vira-A)
zalcitabine (ddC)
zidovudine (Azidothymidine, AZT)

Protease inhibitors
indinavir (Crixivan)
ritonavir (Norvir)
saquinavir (Fortovase)

### Oncologic and Immunological Disorders Breast Cancer

Lung Cancer

Colorectal Cancer

Prostate Cancer

Malignant Lymphomas

Ovarian Cancer

Acute Leukemias

Chronic Leukemias

Melanoma and other Skin Cancers

Hematopoeitic Stem Cell Transplantation

Anti-Neoplastic Medicaments
Alkylating Agents
   busulfan (Myleran)
   carboplatin (Paraplatin)
   carmustine (BNCU, BiCNU)
   cisplatin (Platinol)
   cyclophosphamide (Cytoxan)
   ifofamide (Ifex)
   lomustine (CCNU, CeeNU)
   mechlorethamine (Mustargen)
   melphalan (Alkeran)
   procarbazine (Matulane)
   thiotepa
Antimetabolites
   folic acid Antagonist
   methotrexate
Purine Antagonists 6-mercaptopurine
   6-thioguanine
Pyrimidine Antagonists
   cytarabine (ARA-C)
   fluorouracil (5-FU)
Hormonal Agents Hormones
   diethylstilbestrol (DES)
   estrogens
   prednisone (Deltas one)
Modulation of Hormone Release & Action Aminoglutethimide
   leuprolide acetate
   tamoxifen (Nolvadex)
Plant Alkaloids
   Vinca Alkaloids
      vinblastine (Velban)
      vincristine (Oncovin)
   Podophyllotoxins
      etoposide (VP-16)
   Other
      docetaxel (Taxotere)
      paclitaxel (Taxol)
Antibiotics
   bleomycin (Blenoxane)
   dactinomycin (Cosmegen)
   daunorubicin (DaunoXome)
   doxorubicin (Adriamycin)
   mitomycin (Mutamycin)
Other Anti-neoplastic Medicaments
   amsacrine (AMSA)
   azathioprine (Imuran)
   capecitabine (Xeloda)
   chlorambucil (Leukeran)
   cyclosporine (Sandimmune, Neoral)
   gemcitabine (Gemzar)
   hydroxyurea (Hydrea)
   mitotane (Sodren)
   mitoxantrone (Novantrone)
   pamidronate (Aredia)

Immunosuppressant Medicaments
15-desoxyspergualin
corticosteroids
cyclosporine
Interferons
Interleukins
mycophenolate mofetil
sirolimus (rapamycin)
tacrolimus
thalidomide

### Nutritional Disorders

Malnutrition, vitamin and mineral deficiencies

Enteral Nutrition

Obesity
orlistat (Xenical)
appetite suppressants
   sympathomimetic stimulants
   amphetamine stimulants

Mineral supplementation
calcium ion
iodine
iron
magnesium ion
phosphorous
potassium ion
selenium
sodium ion
zinc

Fat-soluble vitamins
vitamin A
vitamin D
vitamin E
vitamin K

Water-soluble vitamins
vitamin C
thiamine (vitamin B1)
riboflavin (vitamin B2)
niacin (vitamin B3)
pyridoxine (vitamin B6)
folate
cyanocobalamin (vitamin B12)

### Medicaments Used to Alleviate Symptoms of Allergic Rhinitis, Upper Respiratory Symptoms, Cough, Mild Aches and Pains

Nasal Decongestants
ephedrine
phenylephrine
phenylpropanolamine
pseudoephedrine

Antihistamines (Histamine-1 receptor antagonists)

Antitussive agents
benzonatate
codeine
dextromethorphan

Expectorants
guaifenesin
iodinated glycerol
terpin hydrate

Xanthines
aminophylline
caffeine
dyphylline
theophylline

Pain relievers
narcotic agonists
NSAIDS
acetaminophen

### Dietary Supplements

Arnica

### Other Drugs

abacavir sulfate
acetazolamide
acetylsalicylic acid
albendazole
allopurinol
amiloride hydrochloride
amitriptyline hydrochloride artemether
atropine sulfate
benznidazole
biperiden hydrochloride
chloroquine phosphate
chlorpheniramine maleate
chlorpromazine hydrochloride
cimetidine
ciprofloxacin hydrochloride
clofazimine
clomiphene citrate
clomipramine hydrochloride
cloxacillin sodium
codeine phosphate
dapsone
didanosine
diethylcarbamazine citrate
digoxin
diloxanide furoate
DL-methionine

### Doxycycline

### Efavirenz

ergometrine maleate
ergotamine tartrate
erythromycin ethyl succinate
ethambutol hydrochloride
ethosuximide
ferrous sulfate
alendronate sodium
amlodipine besylate
amphetamine (mixed salts)
atorvastatin calcium
benazepril hydrochloride
bisoprolol fumarate
bupropion hydrochloride
carbidopa
cefprozil
cetirizine hydrochloride
citalopram hydrobromide
clindamycin hydrochloride
clonidine hydrochloride
clopidogrel bisulfate
cyclobenzaprine hydrochloride
desloratadine
digoxin
diltiazem hydrochloride
doxazosin mesylate
doxycycline
enalapril maleate
fexofenadine hydrochloride
fluoxetine hydrochloride
folic acid
fosinopril sodium
hydrocodone bitartrate
hydrocodone
hydroxyzine hydrochloride
indinavir
irbesartan
isosorbide mononitrate
lamivudine
levothyroxine sodium
lopinavir
loratadine
losartan potassium
meclizine hydrochloride
medroxyprogesterone acetate
meperidine
metformin hydrochloride
methylphenidate hydrochloride
methylprednisolone
metoclopramide hydrochloride)
minocycline hydrochloride
montelukast sodium
naproxen sodium
nelfinavir
nevirapine
niclosamide
nicotinamide
nifurtimox
nitrofurantoin
nortriptyline hydrochloride
oxybutynin chloride
oxycodone hydrochloride
paracetamol
paroxetine hydrochloride
penicillin V potassium
phenytoin sodium
pioglitazone hydrochloride
prednisolone
primaquine phosphate
pravastatin sodium
prednisolone
promethazine hydrochloride
promethazine fumarate
propylthiouracil
pyrantel embonate
pyridostigmine bromide
raloxifene hydrochloride
ranitidine hydrochloride
rifampicin
risedronate sodium
risperidone
rosiglitazone maleate
salbutamol sulfate
saquinavir mesylate
sertraline hydrochloride
sildenafil citrate
sulfadiazine
sumatriptan succinate
tamoxifen citrate
tamsulosin hydrochloride
temazepam
terazosin hydrochloride
timolol maleate
tolterodine tartrate
tramadol hydrochloride
trazodone hydrochloride
triclabendazole
valacyclovir hydrochloride
valdecoxib
valproic acid
valsartan
venlafaxine hydrochloride
verapamil hydrochloride
warfarin sodium
zolpidem tartrate

Bilberry

Black Cohosh

Cat's claw

Chamomile

Echinacea

Evening Primrose Oil

Fenugreek

Flaxseed

Feverfew

Garlic

Ginger root

Ginko biloba

Ginseng

Goldenrod

Hawthorn

Kava-Kava

Licorice

Milk thistle

Psyllium

Rauwolfia

Senna

Soybean

St. John's wort

Saw palmetto

Turmeric

Valerian

Therapeutic Proteins and Biotechnology Medicaments

What is claimed is:

1. A multi-compartment capsule comprising a therapeutically effective amount of a HMG CoA Reductase inhibitor and a therapeutically effective amount of a serotonin reuptake inhibitor in a single dosage form.

2. A multi-compartment capsule as defined in claim 1, wherein said effective amount of said HMG CoA Reductase inhibitor is between about 20 mg and 100 mg.

3. A multi-compartment capsule as defined in claim 1, wherein said HMG CoA Reductase inhibitor comprises atorvastatin.

4. A multi-compartment capsule as defined in claim 1, wherein said effective amount of said serotonin reuptake inhibitor is between about 10 mg and 300 mg.

5. A multi-compartment capsule as defined in claim 1, wherein said serotonin reuptake inhibitor comprises sertraline.

6. A method for treating hyperlipidemia and depression by administering a therapeutically effective amount of a HMG CoA Reductase inhibitor and a therapeutically effective amount of a serotonin reuptake inhibitor in a single dosage form.

7. A method for treating hyperlipidemia as defined in claim 6, wherein said effective amount of said HMG CoA Reductase inhibitor is between about 20 mg and 100 mg.

8. A method for treating hyperlipidemia as defined in claim 6, wherein said HMG CoA Reductase inhibitor comprises atorvastatin.

9. A method for treating depression as defined in claim 6, wherein said effective amount of said serotonin reuptake inhibitor is between about 50 mg and 300 mg.

10. A method for treating depression as defined in claim 6, wherein said serotonin reuptake inhibitor comprises sertraline.

11. A multi-compartment capsule comprising a therapeutically effective amount of a thiazolidinedione derivative and a therapeutically effective amount of a serotonin reuptake inhibitor in a single dosage form.

12. A multi-compartment capsule as defined in claim 11, wherein said effective amount of said thiazolidinedione derivative is between about 4 mg and 45 mg.

13. A multi-compartment capsule as defined in claim 11, wherein said thiazolidinedione derivative comprises rosiglitazone.

14. A multi-compartment capsule as defined in claim 11, wherein said effective amount of said serotonin reuptake inhibitor is between about 50 mg and 300 mg.

15. A multi-compartment capsule as defined in claim 11, wherein said serotonin reuptake inhibitor comprises paroxetine.

16. A process for treating diabetes and depression by administering a therapeutically effective amount of a thiazolidinedione derivative and a therapeutically effective amount of a serotonin reuptake inhibitor in a single dosage form.

17. A process for treating diabetes as defined in claim 16, wherein said effective amount of said thiazolidinedione derivative is between about 4 mg and 45 mg.

18. A process for treating diabetes as defined in claim 16, wherein said thiazolidinedione derivative comprises rosiglitazone.

19. A process for treating depression as defined in claim 16, wherein said effective amount of said serotonin reuptake inhibitor is between about 50 mg and 300 mg.

20. A process for treating depression as defined in claim 16, wherein said serotonin reuptake inhibitor comprises paroxetine.

21. A multi-compartment capsule comprising a therapeutically effective amount of a bisphosphonate and a therapeutically effective amount of a HMG CoA Reductase inhibitor in a single dosage form.

22. A multi-compartment capsule as defined in claim 21, wherein said effective amount of said bisphosphonate is between about 5 mg and 70 mg.

23. A multi-compartment capsule as defined in claim 21, wherein said bisphosphonate comprises alendronate.

24. A multi-compartment capsule as defined in claim 21, wherein said effective amount of HMG CoA Reductase inhibitor is between about 5 mg and 100 mg.

25. A multi-compartment capsule as defined in claim 21, wherein said HMG CoA Reductase inhibitor comprises simvastatin.

26. A process for treating osteoporosis and hyperlipidemia by administering a therapeutically effective amount of a bisphosphonate with a therapeutically effective amount of a HMG CoA Reductase inhibitor in a single dosage form.

27. A process for treating osteoporosis as defined in claim 26, wherein said effective amount of bisphosphonate is between about 5 mg and 70 mg.

28. A process for treating osteoporosis as defined in claim 26, wherein said bisphosphonate comprises alendronate.

29. A process for treating hyperlipidemia as defined in claim 26, wherein said effective amount of HMG CoA Reductase inhibitor is between about 5 mg and 100 mg.

30. A process for treating hyperlipidemia as defined in claim 26, wherein said HMG CoA Reductase inhibitor comprises simvastatin.

31. A multi-compartment capsule comprising a therapeutically effective amount of a beta-adrenergic receptor antagonist and a therapeutically effective amount of a proton pump inhibitor in a single dosage form.

32. A multi-compartment capsule as defined in claim 31, wherein said effective amount of said beta-adrenergic receptor antagonist is between about 25 mg and 300 mg.

33. A multi-compartment capsule as defined in claim 31, wherein said beta-adrenergic receptor antagonist comprises metoprolol.

34. A multi-compartment capsule as defined in claim 31, wherein said effective amount of said proton pump inhibitor is between about 20 mg and 160 mg.

35. A multi-compartment capsule as defined in claim 31, wherein said proton pump inhibitor comprises omeprazole.

36. A process for treating hypertension and gastroesophageal reflux by administering a therapeutically effective amount of a beta-adrenergic receptor antagonist and a therapeutically effective amount of a proton pump inhibitor in a single dosage form.

37. A process for treating hypertension as defined in claim 36, wherein said effective amount of said beta-adrenergic receptor antagonist is between about 25 mg and 300 mg.

38. A process for treating hypertension as defined in claim 36 wherein said beta-adrenergic receptor antagonist comprises metoprolol.

39. A process for treating gastroesophageal reflux as defined in claim 36, wherein said effective amount of said proton pump inhibitor is between about 20 mg and 160 mg.

40. A process for treating gastroesophageal reflux as defined in claim 36, wherein said proton pump inhibitor comprises omeprazole.

41. A multi-compartment capsule comprising a therapeutically effective amount of an acetylcholinesterase inhibitor and a therapeutically effective amount of an angiotensin II receptor antagonist in a single dosage form.

42. A multi-compartment capsule as defined in claim 41, wherein said effective amount of said acetylcholinesterase inhibitor is between about 5 mg and 50 mg.

43. A multi-compartment capsule as defined in claim 41, wherein said acetylcholinesterase inhibitor comprises rivastigmine.

44. A multi-compartment capsule as defined in claim 41, wherein said effective amount of said angiotensin II receptor antagonist is between about 20 mg and 360 mg.

45. A multi-compartment capsule as defined in claim 41, wherein said angiotensin II receptor antagonist comprises valasartan.

46. A process for treating Alzheimer's disease and hypertension by administering a therapeutically effective amount of an acetylcholinesterase inhibitor and a therapeutically effective amount of an angiotensin II receptor antagonist in a single dosage form.

47. A process for treating Alzheimer's disease as defined in claim 46, wherein said effective amount of said acetylcholinesterase inhibitor is between about 5 mg and 50 mg.

48. A process for treating Alzheimer's disease as defined in claim 46, wherein said acetylcholinesterase inhibitor comprises rivastigmine.

49. A process for treating hypertension as defined in claim 46, wherein said effective amount of said angiotensin II receptor antagonist is between about 20 mg and 360 mg.

50. A process for treating hypertension as defined in claim 46, wherein said angiotensin II receptor antagonist comprises valasartan.
